(19) United States
(12) Patent Application Publication
(10) Pub. No.: US 2022/0193233 A1
(43) Pub. Date: Jun. 23, 2022

(54) STABILIZED ARTIFICIAL IMMUNE COMPLEX ACTIVE IMMUNIZATION STRATEGY THAT SUPPORTS B CELL AND DENDRITIC CELL PROGRAMMING FOR CANCER IMMUNOTHERAPY

(71) Applicants: Thomas McFarland, Albuquerque, NM (US); Greggory May, Paradise Valley, AZ (US); Hugh Mason, Phoenix, AZ (US); Andrew Diamos, Tempe, AZ (US)

(72) Inventors: Thomas McFarland, Albuquerque, NM (US); Greggory May, Paradise Valley, AZ (US); Hugh Mason, Phoenix, AZ (US); Andrew Diamos, Tempe, AZ (US)

(21) Appl. No.: 17/541,273

(22) Filed: Dec. 3, 2021

Related U.S. Application Data
(60) Provisional application No. 63/199,056, filed on Dec. 4, 2020.

(51) Int. Cl. A61K 39/395; C07K 16/32

(52) U.S. Cl. CPC A61K 39/3955; A61K 2039/505; C07K 16/32

(57) ABSTRACT

Breast cancer is a leading cancer diagnosed in women in the U.S. and globally. To combat this deadly disease, continued innovation in immunotherapy treatment methods as an alternative to chemotherapies is gaining traction with promising results. Immunotherapy is the use of medicines to stimulate a subject's own immune system to recognize and destroy cancer cells more effectively. The instant application discloses an immunotherapy treatment and treatment method for breast cancer tumors with Her-2/neu overexpression.

### BACKGROUND

This application claims priority to pending U.S. Provisional Patent Application No. 63/199,056 filed on Dec. 3, 2020, the entire contents of which are incorporated herein by reference.

### SUMMARY

Implementations of the present disclosure are generally directed to focused cancer immunotherapy and related treatment methods. Particularly, implementations of the present disclosure are directed to stabilized artificial immunocomplexes—comprising immunoglobulin protein molecules (antibody) genetically fused to oncogenic protein molecules (oncogen) with linker peptides. A general implementation of the present disclosure involves the deployment of said stabilized artificial immunocomplexes to program B Lymphocytes (B cells) and dendritic cells effective to activate endogenous immune response to oncogenic protein molecules expressed by cancerous tumor cells. [Engeroff et al, (2017)].

In some embodiments, the disclosed treatment method is employed to treat cancers that overexpress the oncogenic protein Her-2/neu using a stabilized artificial immune complex active immunization strategy that supports B cell programming of epitope specific immunoglobulins, which leads to the creation of Her-2/neu specific B memory cells. In some embodiments, the disclosed treatment method supports dendritic cell programming of epitope specific immunoglobulins leading to Her-2/neu T Cell activation and immune response. [Leon, et al (2014)].

In some embodiments, B cells programmed by the stabilized artificial immunocomplexes of the disclosed treatment method have the capacity to express tumor-specific oncogen on its cell surface; and further, produce and secrete the antibody portion of the programming immunocomplexes into a fluid of a subject. In some embodiments, expressed tumor-specific oncogen is available to activate T Cell-based immune response in a subject. In some embodiments, secreted antibodies are available to bind proteins that match the oncogen portion of the immunocomplexes with specificity when expressed by cancerous tumor cells or when encountered in fluid or in tissue—thereby neutralizing their oncogenic properties. Moreover, in some embodiments, programmed B cells can proliferate into B cells with memory when an immune response is triggered by the presence and detection of the oncogen portion of the immunocomplexes. [Daniels, et al. (2003), Engeroff et al. (2017), Lapointe et al. (2003)].

In some embodiments, dendritic cells programmed by the stabilized artificial immunocomplexes of the disclosed treatment method have the capacity to express tumor-specific oncogen on its cell surface. In some embodiments, expressed tumor-specific oncogen is then available to activate T Cell-based immune response in a subject. [Leon, et al (2014)].

In some embodiments, the stabilized artificial immune complexes (ICs) of the present disclosure comprise direct and stable products of immunological recognition by humoral immunity. In some embodiments, these ICs support humoral immune response to cancer in a subject by programming activate B cells and dendritic cells.

In general, ICs can initiate B cell activation by binding to low affinity receptor sites on the B cell surface. Interleukins or helper T Lymphocytes (T cells) costimulate B cells to complete B cell activation. In most cases, both oncogen and a costimulator are required to activate a B cell and initiate B cell proliferation. B cells proliferate and differentiate into plasma cells. The plasma cells produce antibody with the identical oncogen specificity as the oncogen expressed by activated B cells. Antibody is released from the plasma cells and circulates throughout the subject, binding to specific oncogen that may be circulating freely or expressed on cell surfaces. Activated B cells produce memory B cells that provide future immunity.

In general, ICs can activate dendritic cells by binding its antibody portion to high affinity receptor sites on the dendritic cell surface. Activated dendritic cells release interleukins that costimulate B cells and initiate T cell activation. Once activated, dendritic cells express oncogen on its surface to activate T cell immune response.

Accordingly in some embodiments, the present disclosure comprises a stabilized artificial immunocomplex comprising a protein molecule genetically fused to an isolated antibody. In some embodiments, the protein molecule comprises a tumor-specific oncogen derived from a tumor sample extracted from a subject. In some embodiments, the immunocomplex comprises SEQ ID NO. 1 (see sequence listing). In some embodiments, the immunocomplex comprises SEQ ID NO. 2 (see sequence listing). In some embodiments the immunocomplex comprises a gene N-terminal signal peptide such as SEQ ID NO. 3 (see sequence listing). In some embodiments, the protein molecule is a Her-2/neu molecule with a full ectodomain comprising SEQ ID NO. 4 (see sequence listing). In some embodiments, the protein molecule is a Her-2/neu molecule with a truncated ectodomain such as SEQ ID NO. 5 (see sequence listing). In some embodiments, Her-2/neu is genetically fused to an isolated antibody with an amino acid tether comprising SEQ ID NO. 6 (see sequence listing). In some embodiments, the Her-2/neu protein molecule is genetically fused, at its domain III or at its domain IV, to the isolated antibody. In some embodiments, the Her-2/neu molecule is genetically fused to the isolated antibody ex vivo. In some embodiments, the Her-2/neu is genetically fused, at a C-terminus of its domain III or its domain IV, to an N-terminus of the isolated antibody. In some embodiments, the isolated antibody comprises an IgE class antibody. In some embodiments, the isolated antibody comprises an ε-heavy chain variable region comprising SEQ ID NO. 7 (see sequence listing) and a light chain variable region comprising SEQ ID NO. 8 (see sequence listing), both of which are similar to those found in the monoclonal antibody trastuzumab. In some embodiments, the isolated antibody comprises an Fc region that binds to a FcεRI (CD23) receptor on B cell, or a FcεRI receptor on a dendritic cell, or a FcεRII receptor on a dendritic cell. In some embodiments, the isolated antibody is a recombinant monoclonal antibody comprising ε-heavy chain variable region SEQ ID NO. 7 and light chain variable region SEQ ID NO. 8. In some embodiments, the isolated antibody is a humanized or chimeric antibody. In some embodiments, the isolated antibody is an antibody fragment. In some embodiments, the immunocomplex is mono-epitopic. In some embodiments, the immunocomplex has similar binding specificity in each arm of the N-terminus of the isolated antibody of the immunocomplex. In some embodiments, the immunocomplex has similar tumor-specific oncogen linked to the complimentary determining region (CDR) of each arm of the N-terminus of the isolated antibody of the immune complex. Additionally, in some embodiments present disclosure comprises a pharmaceutical composition. In some embodiments, the pharmaceutical composition comprises the stabilized artificial immunocomplex. In some embodiments the stabilized artificial immunocomplex of the pharmaceutical composition comprises a protein molecule genetically fused to an isolated antibody. In some embodiments, the pharmaceutical composition comprises a pharmaceutically acceptable carrier. Additionally, the present disclosure comprises a method for treating a malignant breast cancer tumor in a subject. In some embodiments, the method comprises administering to the subject a therapeutically effective amount of the pharmaceutical composition. In some embodiments, the malignant breast cancer tumor overexpresses Her-2/neu.

In a general implementation of the present disclosure, systems, apparatus, and methods to program B cells with a stabilized artificial immunocomplex (IC) include: designing and administering a stabilized, artificial IC into a subject to promote the binding of the stabilized, artificial IC to a B cell at the B Cell's low affinity receptor site; designing and administering a stabilized, artificial IC into a subject to promote endocytosis of the full stabilized, artificial IC intact into the B cell; designing and administering a stabilized, artificial IC into a subject to promote transport of the oncogenic portions of the stabilized artificial IC to B cell sites that initiate B cell activation; designing and administering a stabilized, artificial IC into a subject to promote the transformation of B cells into effective agents that stimulate the subject's endogenous immune response to overexpressed Her-2/neu without triggering autoimmunity.

In a general implementation of the present disclosure, systems, apparatus, and methods to program dendritic cells with stabilized artificial ICs include: designing and administering a stabilized, artificial IC into a subject to promote the binding of the stabilized, artificial IC to a dendritic cell high affinity receptor site; designing and administering a stabilized, artificial IC into a subject to promote endocytosis of the stabilized, artificial IC into a dendritic cell; designing and administering a stabilized, artificial IC into a subject to promote interleukin secretion from a dendritic cell into the fluid of a subject to support B cell costimulation and T cell activation; designing and administering a stabilized, artificial IC into a subject to promote transport of oncogenic portions of the stabilized artificial IC to dendritic cell sites that initiate T cell immune response; designing and administering a stabilized, artificial IC into a subject to promote the transformation of dendritic cells into effective agents that stimulate the subject's endogenous immune response to overexpressed Her-2/neu without triggering autoimmunity.

An aspect combinable with the general implementation further includes stabilized artificial ICs programming B cells that differentiate into plasmid cells specialized to generate IC-comprising antibody tailored to bind and neutralize IC-comprising oncogen expressed by cancerous tumor cells with binding specificity.

In an aspect combinable with any of the previous aspects, stabilized artificial ICs program B cells that produce memory B cells with the capacity to differentiate into specialized plasmid cells upon re-exposure to the IC-comprising oncogen.

It is appreciated that methods in accordance with the present disclosure can include any combination of the aspects and features described herein. That is, methods in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

FIG. 1 depicts an example of an artificial immunocomplex comprising an IgE immunoglobulin that is genetically fused to a specific oncogen (such as Her-2/neu) at its epitope.

FIG. 2 depicts an example IgE immunoglobulin molecule comprising in planta glycosylation of IgE to target low affinity CD23 IgE receptor on B-cells

FIG. 3 depicts an example designed Her-2/neu oncogenic protein molecule comprising a full ectodomain (Domain I-IV) and a Her-2/neu oncogenic protein comprising a truncated domain III and IV.

FIG. 4 depicts an example designed Her-2/neu oncogenic protein molecule interacting with an example IgE immunoglobulin to form a stabilized artificial IC.

FIG. 5 depicts the example artificial recombinant IC comprising an IgE immunoglobulin protein molecule genetically fused to a truncated Her-2/neu oncogenic protein molecule at its N-terminus of IgE corresponding to CDR specific hypervariable regions.

FIG. 6A depicts Her-2/neu with truncated Domain III and Domain IV from the full ectodomain.

FIG. 6B depicts an IgE/Her-2/neu immunocomplex comprising Her-2/neu with truncated Domain III and Domain IV genetically fused to an IgE monoclonal antibody with a peptide linker.

FIG. 7 depicts a recombinant specific IgE/antigen immune complex interacting with B cell- and dendritic cell-mediated immune response pathways.

FIG. 8 depicts a programmable network of stimulation and costimulation of B Cells and Dendritic cells by an IgE/Her-2/neu immune complex.

FIG. 9 depicts two IgE/Her-2 Immune complex B cell-mediated pathways to programming.

FIG. 10 depicts a cancer tumor generating a microenvironment (stroma) that inhibits immune response surveillance.

FIG. 11 depicts a tumor-generated stroma causing immune response suppression by blocking tumor antigen processing by immune complexes or antigen-presenting cells.

FIG. 12 depicts a cancer immunity cycle whereby the immune complex of the disclosure triggers immune response to tumor antigen by stimulating the production of immunoglobulins specific to cancer tumor antigen.

FIG. 13 depicts a method to reactivate a cancer immunity cycle whereby the immune complex of the disclosure is introduced into a subject to trigger immune response to tumor antigen by stimulating the production of immunoglobulins specific to cancer tumor antigen. Designed IgE/Her-2 ex-vivo immune complexes reactivate tumor immune response through B cell and dendritic cell programming. Once B-cell CD-23 mediated endocytosis occurs within the lymph node or other tertiary lymphoid tissues it reactivates tumor immune response programming and tumor stromal infiltrating lymphocyte migration to the tumor and stroma. This reactivation of lymphocyte migration to the tumor tissue includes tumor specific antigen sensitized B-cells, dendritic cells, CD4+ and CD+T− cells and specific immunoglobulins. The stroma and the tumor tissue then becomes the extended active site of the anti-tumor immune response. [Alberts, et al. (2021)]

FIG. 14 depicts a table showing that the IgE/Her 2/neu immunocomplex binds human CD23 (low affinity receptor) with greater affinity than human IgE alone.

FIG. 15 depicts a IgE/Her-2 Immune Complex Plasmid featuring Her 2/neu with full ectodomain.

FIG. 16 depicts a IgE/Her-2 Immune Complex Plasmid featuring Her 2/neu with truncated domain III and IV.

### DETAILED DESCRIPTION

Cancer immunotherapies such as antibodies targeting T cell checkpoints, or adaptive tumor-infiltrating lymphocyte (TIL) transfer, can be used to boost immune response against human malignancies. However, activation of T cells by such antibodies can lead to the risk of autoimmune diseases. Moreover, the selection of tumor-reactive T cells for TIL relies on information regarding mutated oncogen in tumors and does not reflect other factors involved in protein oncogenicity. Therefore, the disclosed treatment method incorporates therapeutic interventions by which T cell reactivity against tumor cells is selectively enhanced (i.e., focused cancer immunotherapy) based on tumor oncogen that are specifically expressed in the tumor of a certain cancer and in many patients with that cancer.

The humanized monoclonal IgG1 antibody trastuzumab (Herceptin®) binds to the extracellular domain of HER-2/neu. Trastuzumab was initially approved in 1998 by the Food and Drug Administration for the treatment of HER-2/neu overexpressing in advanced breast cancers. [Ahn and Vogel (2012)]. Since then, trastuzumab has shown efficacy against breast cancer both as an adjuvant therapy and as a treatment of metastatic disease. However, most patients with advanced breast cancer that are treated with trastuzumab alone or combined with chemotherapeutic agents eventually relapse and the median time to progression is less than 1 year. Additionally, a significant number of breast cancer patients do not respond to trastuzumab-based therapies despite the high level of HER-2/neu expression. Furthermore, in a Phase II clinical trial in patients with HER-2/neu overexpressing recurrent or refractory ovarian or primary peritoneal carcinoma that were treated with trastuzumab alone, a low rate of objective response (7.3%) was observed. [Bookman et al. (2003)]. While trastuzumab has shown efficacy in a subset of patients with either breast or ovarian cancer, additional strategies to target HER-2/neu overexpressing tumors are still needed.

Like trastuzumab, most antibody therapies for the treatment of cancer utilize antibodies that are of the IgG class. However, antibodies of the IgE class may also be potential cancer therapeutics since they have several potential advantages over their IgG counterparts. IgE mediates allergic reactions, which is due to the presence of effector cells in the tissue that are sensitized by IgE bound to Fc epsilon receptor I (FcεRI). These effector cells are degranulated after crosslinking of the IgE that is triggered by a multi-epitope oncogen interaction. IgE can also mediate oncogen presentation via the interaction with FcεRs expressed on oncogen-presenting cells (APC) such as dendritic cells (DC). IgE has been suggested to provide protection against parasitic infections, although this function is controversial. Research on cancer and IgE belongs to the new field of onco-immunology. This field has two aims: (1) to reveal the function of IgE-mediated immune responses against cancer cells to elucidate the understanding of its biology and (2) to develop novel IgE-based treatment options against malignant diseases. [Jensen-Jarolim et al. (2019)]. A key advantage associated with IgE is its exceptionally high affinity for the FcεRs. There are two FcεRs, the FcεRI which binds IgE with high affinity (with an association constant on the order of 1010 M⁻¹) and is expressed on human dendritic cells; and the FcεRII (CD23) which binds IgE with lower affinity (with an association constant on the order of 108 M⁻¹) and is expressed on human B cells. [Chauhan et al. (2020)]. Thus, the affinity of IgE for FcεRI is at least two orders of magnitude higher than that of IgG for the FcγRs (FcγRI-III) and in the case of FcεRII is as high as that of IgG for its high-affinity receptor FcγRI (CD64). [Chauhan et al. (2020)]. Another advantage of the IgE molecule is the low endogenous serum concentration in humans, which is only 0.02% of total circulating immunoglobulins, whereas IgG is the most abundant at 85%. [Chauhan et al. (2020)]. Thus, the competition for FcR occupancy is much lower for IgE. Another potential advantage is that there is no known inhibitory FcεR as there is for FcγR.

Fully human anti-HER-2/neu IgE has been developed and evaluated for its potential as a cancer therapeutic. After an immunogenic stimulation in a subject, antibodies are produced by specialized B cells for the purpose of combining with the evoking oncogenic determinants wherever they are encountered, thereby forming immune complexes (ICs). This process is part of a humoral immune response and is usually of benefit to the subject because it leads to the neutralization or elimination of the oncogen.

ICs act as regulatory factors in immune responses because they can interact with oncogen receptor-bearing lymphocytes and subpopulations of T and B cells, as well as unclassified lymphocytes and macrophages having Fc and complement (C) receptors. ICs can modulate humoral and cellular immune responses by interacting with B and T cells having Fc, C, and/or oncogen receptors. Through such interactions, ICs may suppress or augment immune responses, depending on the molar ratio of the oncogen and antibody, the epitope density of the complex, the steric and chemical conformation of oncogene, and the mass, class, and affinity of antibody.

In some embodiments, the formation and function of ICs according to aspects of the disclosure treatment method include two features. First, the immunoglobulin (Ig) class, which determines antibody valence for a specific oncogen as well as its ability to bind to cellular Fc receptors and to activate the C system, is IgE. Second, the association constant for the union of specific antibody and oncogen is sufficient to form a stabilized IC. Generally, B cells have receptors for IgE Fc. Therefore, in some embodiments, the artificial ICs of this disclosure, comprising IgE immunoglobulin bind to B cells at Fc sites. In some embodiments, IgE binding to B cells at its Fc site program B cells, which can act as effective agents to activate endogenous immune response when introduced within a subject without triggering autoimmunity.

Accordingly, implementations of the present disclosure are generally directed to focused cancer immunotherapy, allergo-oncology, and related treatment methods. Particularly, implementations of the present disclosure are directed to stabilized artificial immunocomplexes—comprising immunoglobulin antibody genetically fused to oncogenic protein (oncogen)—that program B Lymphocytes (B cells) and dendritic cells, effective to activate endogenous immune response to oncogenic protein expressed by cancerous tumor cells or encountered in a subject fluid.

In one implementation, the stabilized artificial immune complexes are directed to focused breast cancer immunotherapy and related treatment methods. Approximately 25% of breast cancers demonstrate amplification of the oncogen HER-2/neu, which is associated with more aggressive disease and poor prognosis. In some studies, HER-2/neu overexpression has also been described in 9-32% of ovarian cancer tissue. [Ahn and Vogel (2012)]. As is the case for breast cancer, HER-2/neu overexpression in ovarian cancer is associated with poor prognosis. [Ahn and Vogel (2012)]. HER-2/neu is a member of the epidermal growth factor receptor (EGFR) family that have intrinsic tyrosine kinase activity that leads to the activation of downstream signaling pathways of cell proliferation and survival. [Ahn and Vogel (2012)].

Implementations of the present disclosure are directed to IgE class immunoglobulins as target proteins to be genetically fused to oncogen in the formation of artificial ICs. Traditionally, immunoglobulin E (IgE) has an evolutionary role in mammals as the primary line of defense against parasites and venoms. [Mukai et al. (2016)]. In this role IgE acquire powerful effector functions to expedite adaptive immune sensitization. [Mukai et al. (2016), Sutton et al. (2019)].

IgE's Fc region receptor-binding is unique among other classes of immunoglobulins. The high affinity receptor FcεRI is structurally homologous to other members of the FcR family and is found on multiple effector cells. The second IgE receptor FcεRII (CD23) is unlike all other antibody receptors. It is a member of the C-type (Ca2+-dependent) lectin-like superfamily [Sutton et al. (2015), Griffith (2011)] and functions to endocytose the entire antigen/immunoglobulin immune complex. CD23 is the target receptor for this immune complex. [Sutton et al. (2019), Karagiannis (2001)].

IgE differs from the other sub-classes of immunoglobulins in its domain architecture, glycosylation, conformational dynamics, and allosteric properties. These attributes are favorable for the endocytosis of the entire antigen/immunoglobulin immune complex. IgE is the most heavily glycosylated member of the immunoglobulin family. [Sutton et al. (2019)]. Manipulation of the heavy chain glycosylation profile in this immune complex allows targeting of the FcεRII (CD23) IgE receptor on the surface of B cells and endocytosis of the antigen/immunoglobulin immune complex. [Sutton et al. (2019), Sutton et al. (2015), Griffith (2011), Karagiannis (2001)]. Glycosylation is functionally important for unloading of IgE/antigen complexes by CD23 in endosomes. [Sutton et al. (2019), Karagiannis (2001)].

Although the FcεRI and FcεRII work in concert and are engaged by the Fc portion of the immunocomplex of the disclosure, the glycosylation sites on the fully constant region are altered to express greater affinity for the unique secondary IgE receptor FcεRII/CD23. The CD23 receptor has the function of transferring immunocomplex from cell surface CD23 receptors on the surface of B cells into non-degradable compartments within the B cell and recycling them as cell surface receptors for immune antigen sensitization interactions with CD4 and CD8 t-cells and dendritic cells. [Karagiannis (2001)]. B cell activation by CD23 endocytosis has also been shown to initiate B cell migration from lymph node and spleen to tumor tissues for sensitization of T cells and production of tumor specific immunoglobulin. [Acharya et al. (2010)].

It has been previously shown that IgE complexed with a mono-epitopic antigen through its interaction with CD23/Fcε RII is capable of mediating antigen presentation in the absence of receptor cross-linking. [Daniels et al. (2012), Bheekha et al. (1995)]. In some embodiments of the instant disclosure, the immunocomplex is mono-epitopic in that it has similar specificity and similar antigen linked to the complimentary determining region (CDR) of each arm of the N-terminus of the immunoglobulin E component of the immunocomplex.

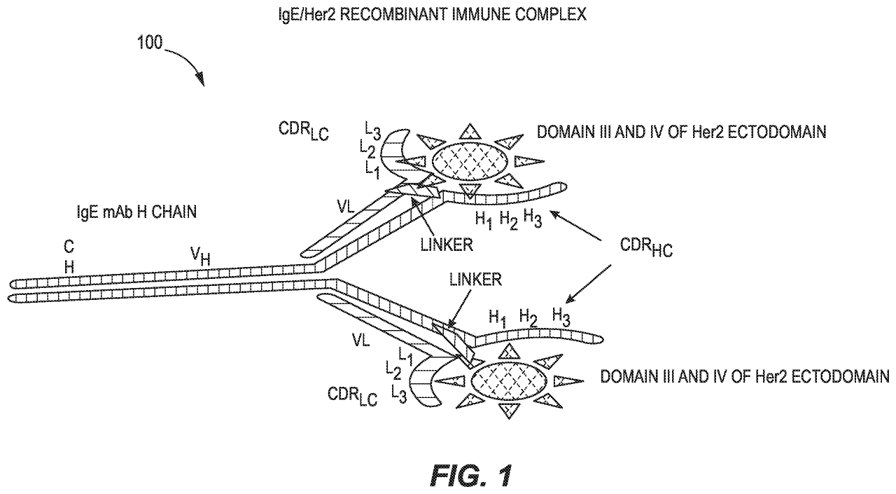

An example artificial recombinant IC 100 comprising an IgE immunoglobulin protein molecule genetically fused to a Her-2/neu oncogenic protein molecule is shown in FIG. 1. In some embodiments, the 100 comprises a twelve amino acid tether used to fuse immunoglobulin to oncogenic protein. In some embodiments, 100 comprises IgE immunoglobulin. In some embodiments, 100 comprises Her-2/neu protein. In some embodiments, 100 is introduced directly into a subject to program B cells existing within the subject—programming sufficient to cause the B cells to act as effective agents to activate endogenous immune response without triggering autoimmunity. In some embodiments, 100 programs B cells outside of the subject. Further, in some embodiments, B cells programming outside of the subject is sufficient to cause the B cells to act as effective agents to activate endogenous immune response when introduced within the subject without triggering autoimmunity.

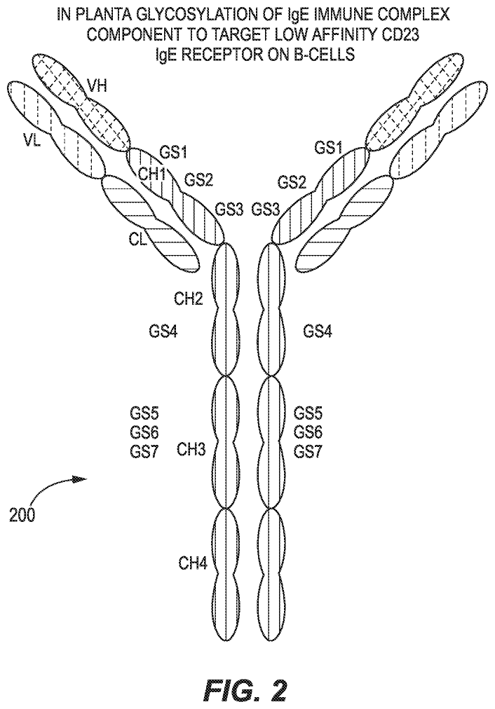

An example IgE immunoglobulin molecule 200 comprising In Planta glycosylation of IgE to target low affinity CD23 IgE receptor on B-cells is shown in FIG. 2. In some embodiments, the example IgE immunoglobulin molecule comprises targeting glycosylation sites GS1-GS7 with >90% GlcNAc terminating glycoforms. [Montero-Morales et al. (2017), Jegouzo et al. (2019)]. In one embodiment of 100, 200 has a higher affinity for the CD23 low affinity IgE receptor than the FcεRI high affinity IgE receptor. Specifically, 200 targets and selectively binds the CD23 low affinity IgE receptor. In some embodiments, this binding is essential for endocytosis of the immune complex to a non-degradable compartment within the B-cell for cycling back to the cell surface to interact with T-cells and dendritic cells. [Engeroff, et al. (2021), Getahun et al. (2005), Griffith et al. (2011), Sutton et al., (2015)].

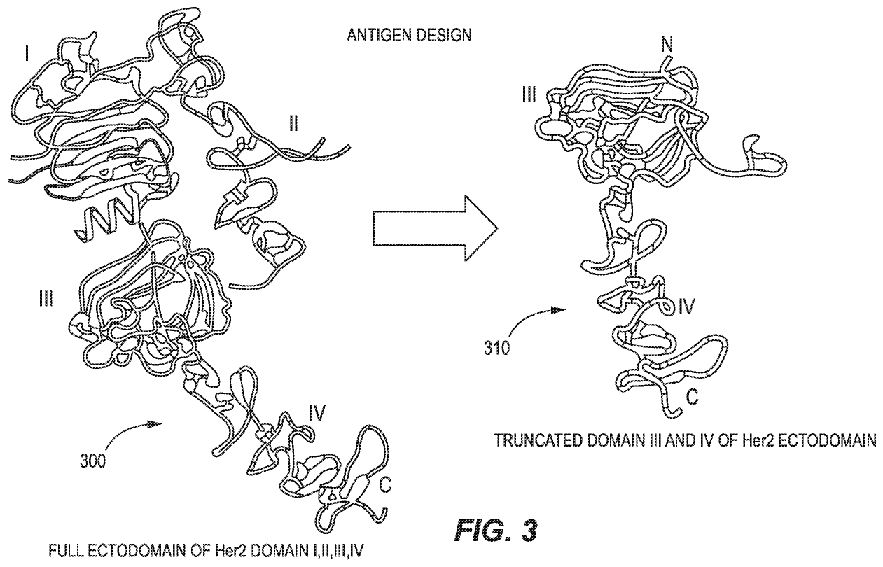

An example of a designed scheme to form a Her-2/neu oncogenic protein molecule comprising a truncated domain III and IV 310 from a Her-2/neu protein with a full ectodomain (Domain I-IV) 300 is shown in FIG. 3. This figure further depicts the exposed N-terminus of the 310. In one embodiment of 100, 310 is genetically fused to 200 at the N-terminus or 310.

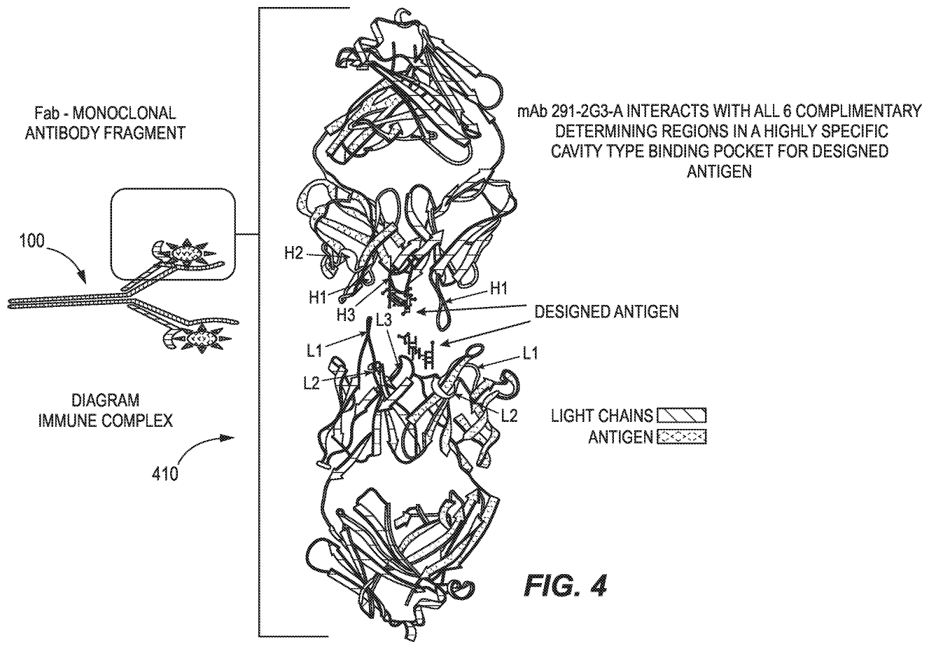

An example designed Her-2/neu oncogenic protein molecule interacting with an example IgE immunoglobulin 410 to form 100 is shown in FIG. 4. In one embodiment, 200 is a monoclonal antibody fragment of type mAb 291-2G3-A which interacts with all 6 complimentary determining regions in a highly specific cavity type binding pocket for 310. FIG. 4. Further depicts light chain regions and Her-2/neu. [Choi et al. (2011), Cho Hyun-Soo et al. (2003)].

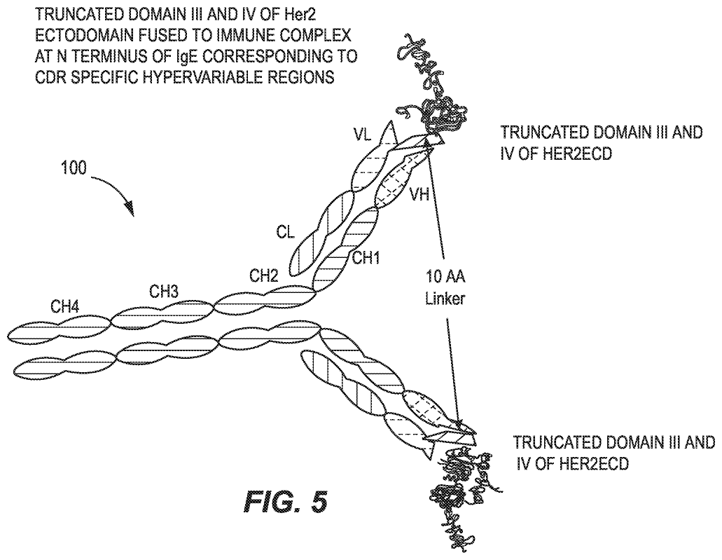

FIG. 5 depicts 100 comprising 310 genetically fused to 200 at the N-terminus of 200 corresponding to CDR specific hypervariable regions. FIG. 5 further depicts a tether of at least ten (10) amino acids that fuses 200 to domain III or IV of 310.

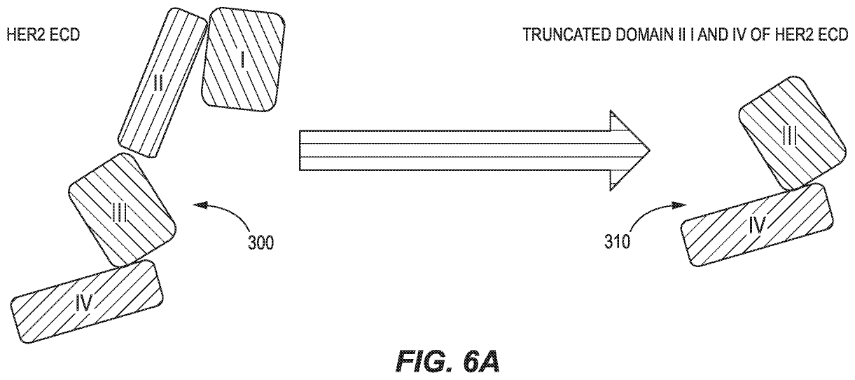

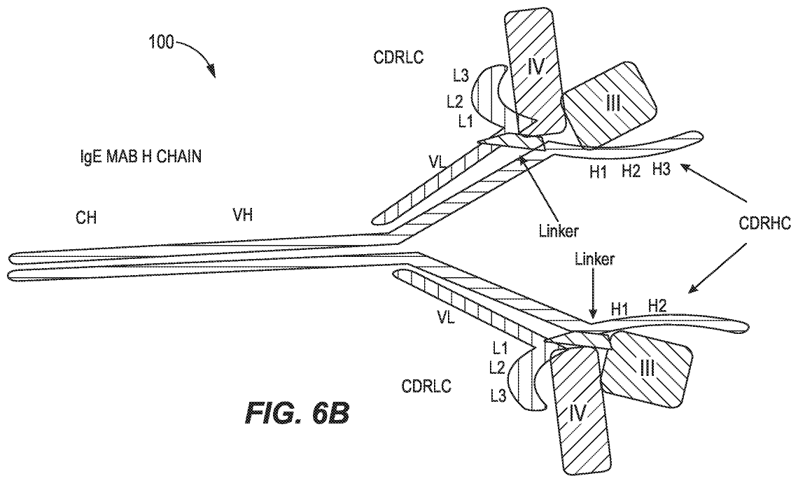

FIG. 6A depicts 310 designed from 300. There is evidence that an epitope spanning domain III and IV enhances an antibody's ability to disrupt HRG-dependent ErbB2/ErbB3 signaling 1. [Fu et al. (2014), Yarden et al. (2001)]. FIG. 6B depicts 100 comprising 310.

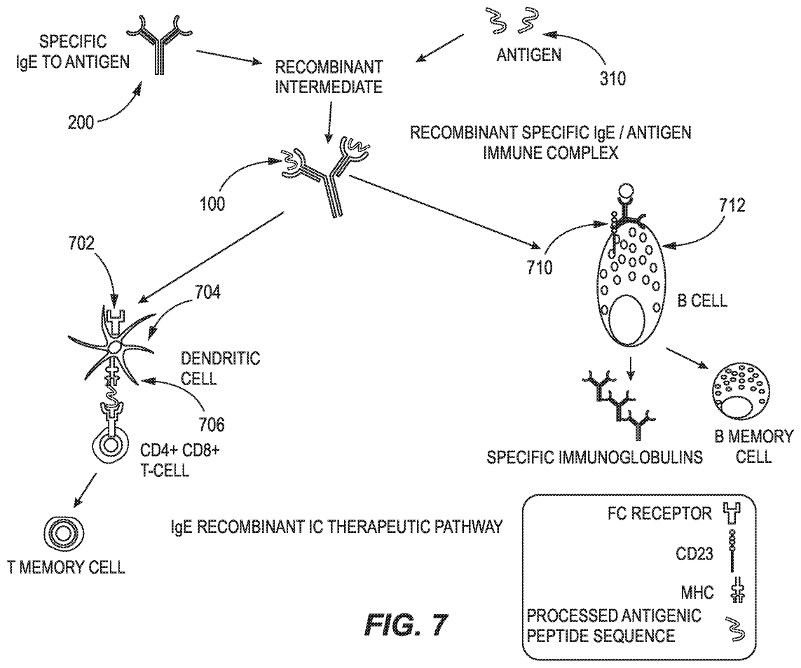

100 can interact with two immune cell-mediated therapeutic pathways as shown in FIG. 7. The Fc portion of 100 binds with Fc receptor sites 702 on dendritic cells 704, activating a dendritic cell-mediated pathway 706 to program CD4+ and CD8+ T cells and memory T cells. [Bergtold, et al. (2005), Martin, et al (2014), Platzer et. al (2014)]. Also, 100 can bind low affinity receptors (CD23) 710 located on the surface with B cells 712, thus activating mediated pathways to program B cell generated 200 that can selectively bind the 300 or 310 within the subject and to program memory B cells. [Alberts, et al. (2021), Nzula (2003), Phan et al. (2007), Phan et al. (2009)].

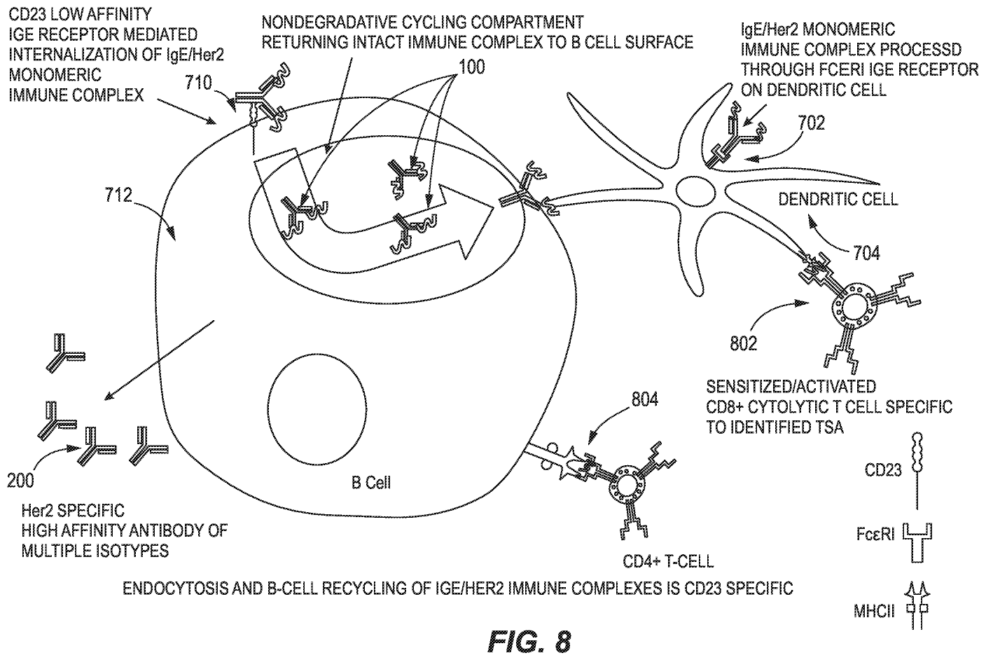

FIG. 8 depicts endocytosis and B-cell recycling of 100 with binding specificity 710. 710 is a C-type lectin low affinity receptor for IgE that is expressed on IC-programmed B cells and performs several functions once activated: (i) present intact 100 at surface of 704 at 702 for endocytosis, (ii) intact antigen presentation via MHCII for CD4+ T-Cell activation 804, and (iii) B cell differentiation to generate 200-producing plasma B cells and B memory cells. FIG. 8 further depicts activated 704 presentation of 300 or 310 via MHCII for CD8+ T-cell activation 802. [Bergtold, et al. (2005) Martin, et al (2014) Platzer, et. al. (2014) León, B. et al (2014)].

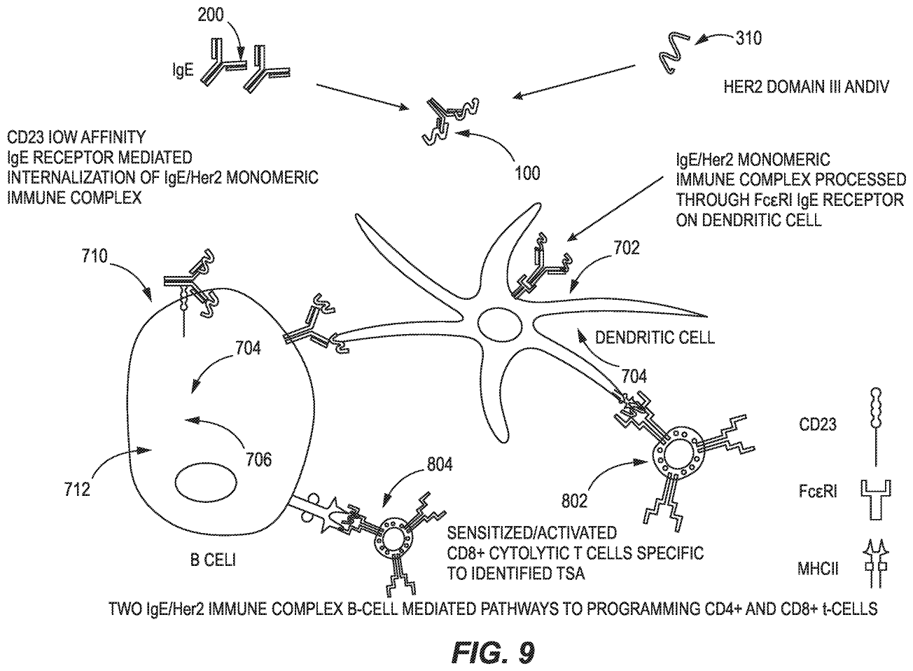

FIG. 9 depicts two IgE/Her-2 Immune complex B cell mediated pathways to programming. In one pathway, 710 located on 712 mediates the intact internalization of 100 into 712. While in another pathway, 100 processed through 702 on 704. Both pathways lead to either sensitized or activated CD8+ cytolytic T cells specific to 300 or 310 of 100.

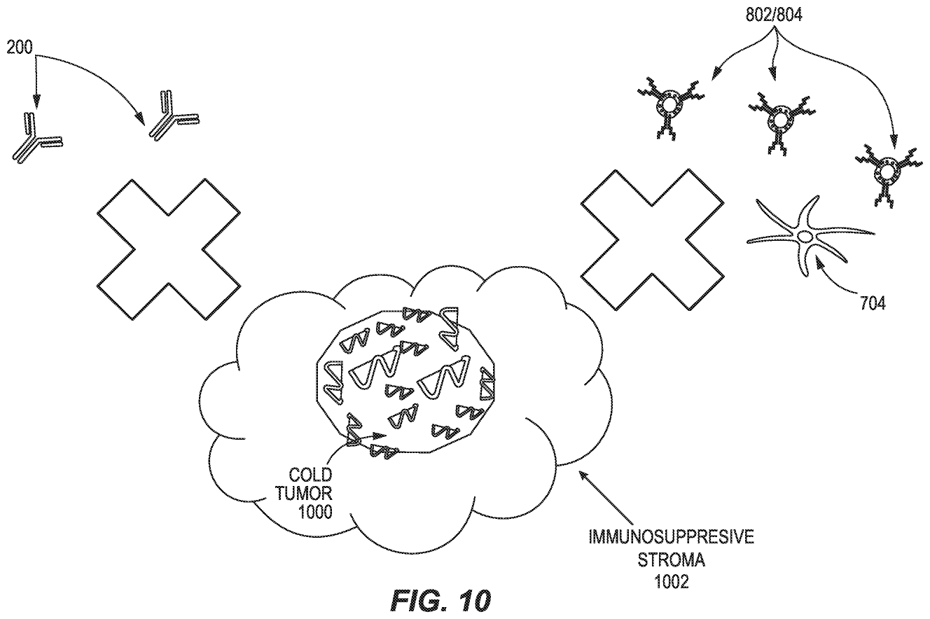

FIG. 10 depicts a cancer tumor 1000 generating a microenvironment (stroma) 1002 that inhibits immune response surveillance (blocks interactivity with 200). Tumor-generated stroma (microenvironment) can inhibit immune surveillance and blocks CD4+ and CD8+ T-cell access through dendritic cell-mediated pathways (704, 802, 804) and immunity cycle by inhibiting antigen processing by immunoglobulins and dendritic cells.

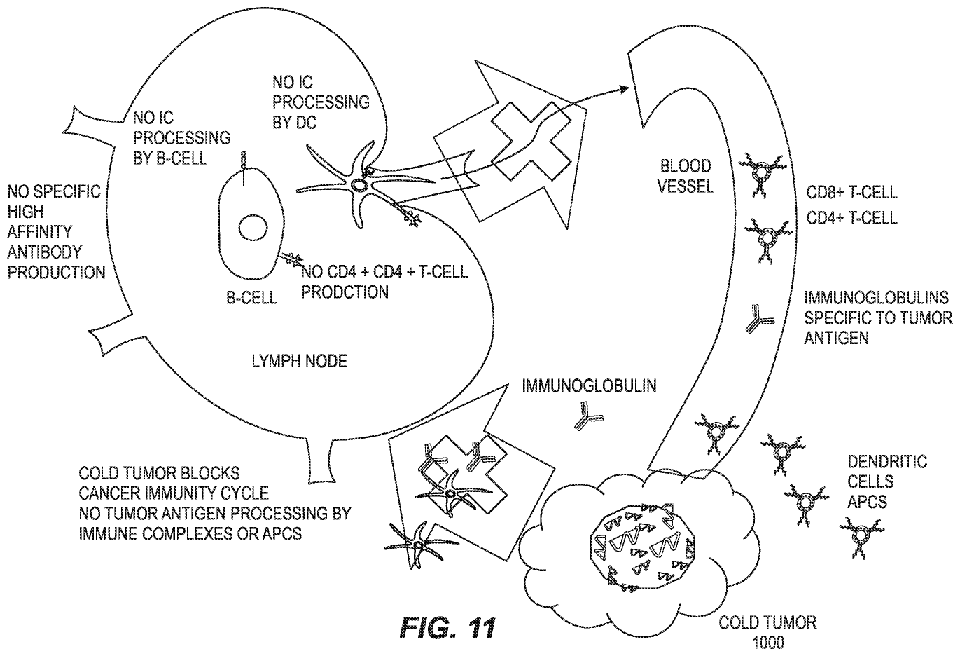

FIG. 11 depicts 1000 causing immune response suppression by blocking 300 processing by 704 and 712 or by antigen-presenting cells. [Whatcott et. al. (2015) Spear et. al. (2019)].

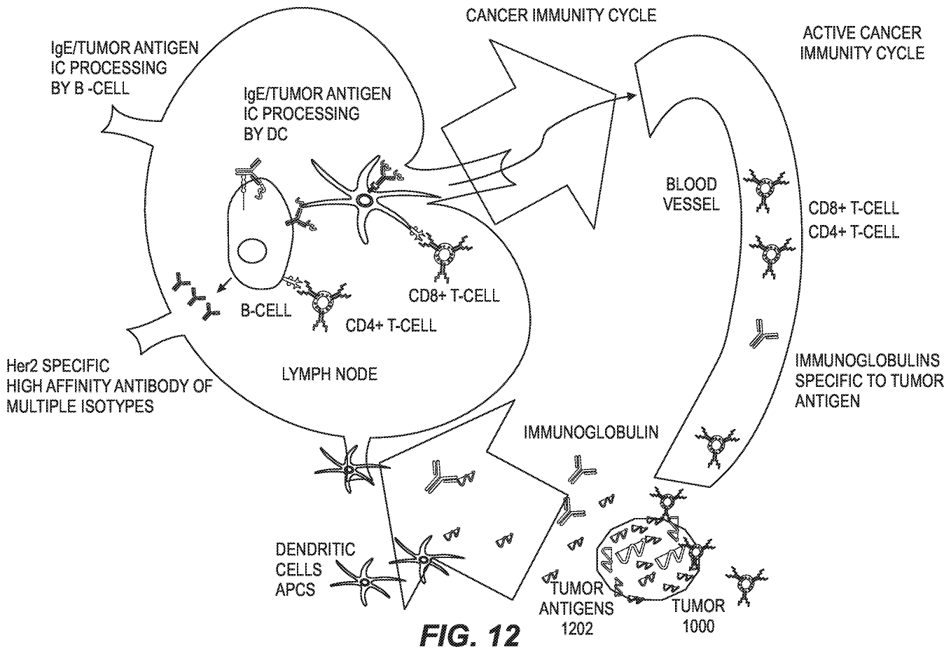

FIG. 12 depicts a cancer immunity cycle whereby 100 of the disclosure triggers immune response to tumor antigens 1202 by stimulating the production of 200 to fight 1000 within the subject. [Lapointe et al. (2003) Nzula, (2003)].

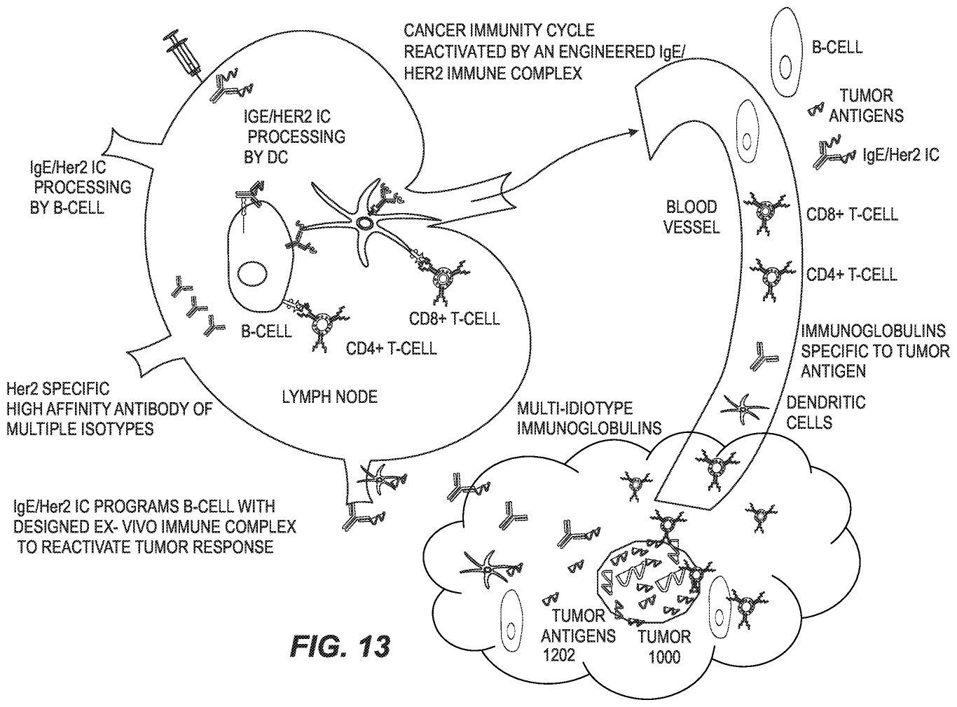

FIG. 13 depicts an active cancer immunity cycle wherein designed 100 ex vivo reactivate tumor immune response through 704 and 712 programming upon administration of 100 to the subject. In some embodiments, 100 triggers immune response to 1202 by stimulating the production of 200. [Lapointe et al. (2003), DiLillo et al. (2009,), Candolfi et al (2011) Alberts, et al. (2021)]. Once 712/710-mediated endocytosis occurs within the lymph node or other tertiary lymphoid tissues it reactivates tumor immune response programming and tumor stromal infiltrating lymphocyte migration to 1000 and 1002. This reactivation of lymphocyte migration to the tumor tissue includes tumor specific antigen sensitized B cells, dendritic cells, CD4+ and CD+ T cells and 200. 1000 and 1002 then become the extended active site of the anti-tumor immune response. [Alberts, et al. (2021), Nzula, (2003)].

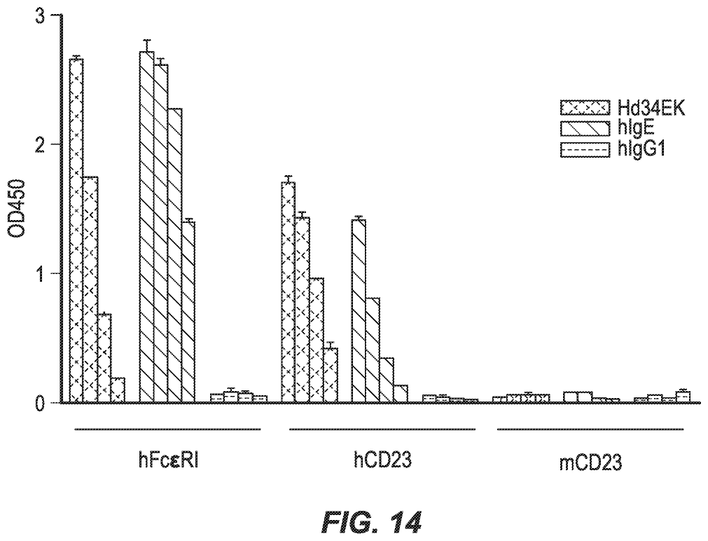

FIG. 14 depicts a table showing that 100 binds 710 with greater affinity than 200 alone. Targeting glycosylation sites GS1-GS7 on 200 with >90% GlcNAc terminating glycoforms results in 100 with higher affinity for 710 than the FcεRI high affinity IgE receptor. Engaging 710 is essential for unique 100 processing by the 712. [Montero-Morales et al. (2017), Jegouzo et al. (2019), Acharya et al. (2010)].

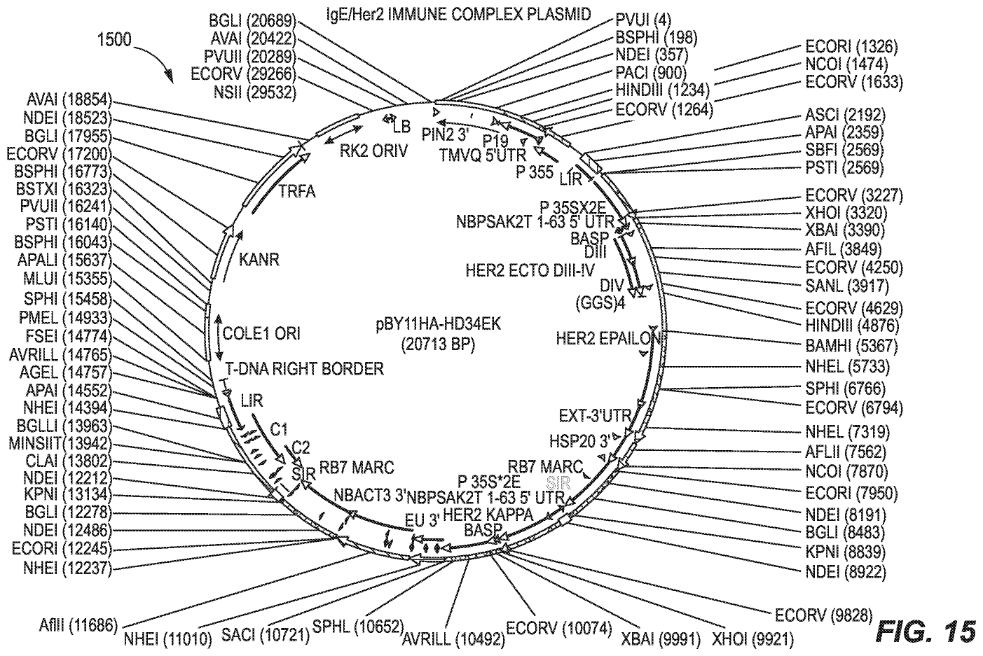

FIG. 15 depicts a plasmid vector map for cloning translational immune complex protein IgE/Her2/neu full ectodomain that can be expressed by N. benthamiana plants or mammalian Chinese hamster ovarian (CHO) intermediates. [Montero-Morales et al., (2019)].

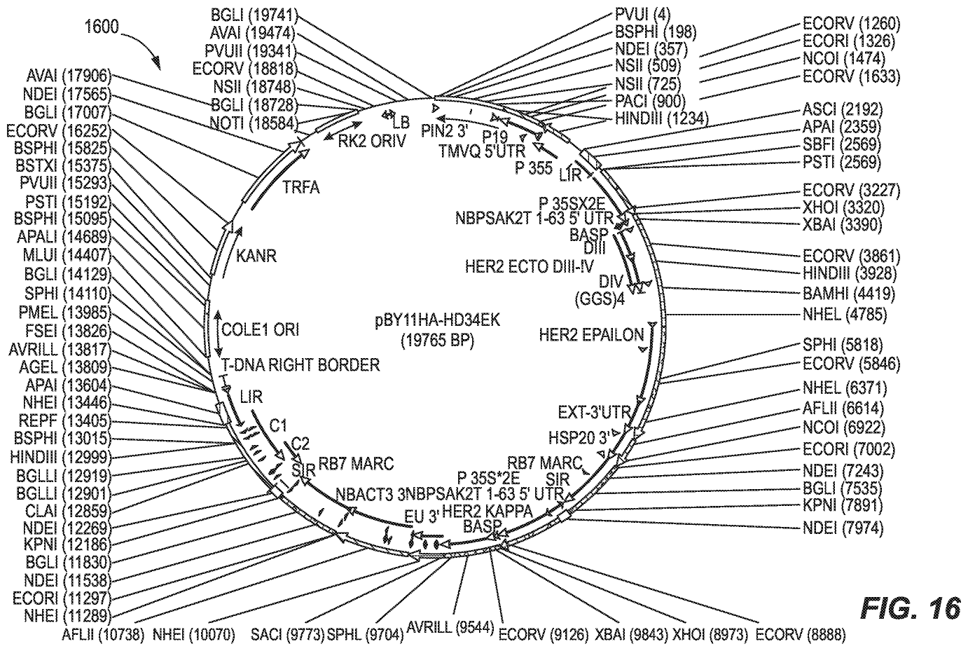

FIG. 16 depicts a plasmid vector map for cloning translational immune complex protein IgE/Her2/neu full ectodomain that can be expressed by N. benthamiana plants or mammalian Chinese hamster ovarian (CHO) intermediates. [Montero-Morales et al., (2019)]

### EXAMPLES

Reference is now made to the following examples, which together with the above descriptions, illustrate embodiments of the disclosure in a non-limiting fashion.

The following examples describe materials and methods to produce a recombinant protein (target protein) comprising an IgE molecule specific for receptor tyrosine-protein kinase erbB-2 (also known as Her-2) [Homo sapiens], that is genetically fused to the same Her-2 antigen. One having ordinary skill in the art will recognize that the fusion protein permits binding of the antigenic epitope of Her-2 by the CDR regions of the antibody, thus making an immune complex. Generally, the nomenclature used herein and the laboratory procedures utilized in the present disclosure include molecular, biochemical, microbiological and recombinant DNA techniques. Such techniques are thoroughly explained in the literature.

### Materials and Experimental Methods

Designing fusion proteins: The target protein comprises an IgE molecule composed of two heavy chains (heavy chain, epsilon type) and two light chains (light chain) assembled in the usual way [see e.g., Diamos et al, (2019)], wherein the antigen combining site is specific for Her-2 antigen, and the heavy chain and light chain variable regions are the same as those found in the monoclonal antibody trastuzumab (Herceptin®) (i.e. PDB: 6OGE_E and 6OGE_D) with a structure similar to that indicated in NCBI Structure Database 6OGE. Further, the target protein comprises Her-2 antigen fused to the variable region of either the heavy chain or light chain of trastuzumab. Using the published structures determined for trastuzumab bound to Her-2 as guides, the instant design discloses several embodiments for genetic fusions including: 1) C-terminus of Her-2 fused to N-terminus of heavy chain or 2) C-terminus of Her-2 fused to N-terminus of light chain, and the use of linker peptides of varying lengths and content of glycine/serine. Other embodiments include 1) use of the full length ectodomain of Her-2 vs. the C-terminal domain IV of Her-2 to which the antibody binds, and 2) use of the native Her-2 ER-targeting signal peptide vs. the barley alpha amylase gene signal peptide that have been used extensively for other recombinant antibodies and antibody fusion proteins.

Fusion Protein Amino Acid Sequence for IgE/her 2/Neu Immune Complex with Full her 2/Neu Ectodomain (SEQ ID NO. 1):

MANKHLSLSLFLVLLGLSASLASGTQVCTGTDMKLRLPASPETHLDM
LRHLYQGCQVVQGNLELTYLPTNASLSFLQDIQEVQGYVLIAHNQVR
QVPLQRLRIVRGTQLFEDNYALAVLDNGDPLNNTTPVTGASPGGLRE
LQLRSLTEILKGGVLIQRNPQLCYQDTILWKDIFHKNNQLALTLIDT
NRSRACHPCSPMCKGSRCWGESSEDCQSLTRTVCAGGCARCKGPLPT
DCCHEQCAAGCTGPKHSDCLACLHFNHSGICELHCPALVTYNTDTFE
SMPNPEGRYTFGASCVTACPYNYLSTDVGSCTLVCPLHNQEVTAEDG
TQRCEKCSKPCARVCYGLGMEHLREVRAVTSANIQEFAGCKKIFGSL
AFLPESFDGDPASNTAPLQPEQLQVFETLEEITGYLYISAWPDSLPD
LSVFQNLQVIRGRILHNGAYSLTLQGLGISWLGLRSLRELGSGLALI
HHNTHLCFVHTVPWDQLFRNPHQALLHTANRPEDECVGEGLACHQLC
ARGHCWGPGPTQCVNCSQFLRGQECVEECRVLQGLPREYVNARHCLP
CHPECQPQNGSVTCFGPEADQCVACAHYKDPPFCVARCPSGVKPDLS
YMPIWKFPDEEGACQPCPINCTHSCVDLDDKGCPAGGSGGSGGSGGS
EVQLVESGGGLVQPGGSLRLSCAASGFNIKDTYIHWVRQAPGKGLEW
VARIYPTNGYTRYADSVKGRFTISADTSKNTAYLQMNSLRAEDTAVY
YCSRWGGDGFYAMDYWGQGTLVTVSSASTQSPSVFPLTRCCKNIPSN
ATSVTLGCLATGYFPEPVMVTWDTGSLNGTTMTLPATTLTLSGHYAT
ISLLTVSGAWAKQMFTCRVAHTPSSTDWVDNKTFSVCSRDFTPPTVK
ILQSSCDGGGHFPPTIQLLCLVSGYTPGTINITWLEDGQVMDVDLST
ASTTQEGELASTQSELTLSQKHWLSDRTYTCQVTYQGHTFEDSTKKC
ADSNPRGVSAYLSRPSPFDLFIRKSPTITCLVVDLAPSKGTVNLTWS
RASGKPVNHSTRKEEKQRNGTLTVTSTLPVGTRDWIEGETYQCRVTH
PHLPRALMRSTTKTSGPRAAPEVYAFATPEWPGSRDKRTLACLIQNF
MPEDISVQWLHNEVQLPDARHSTTQPRKTKGSGFFVFSRLEVTRAEW
EQKDEFICRAVHEAASPSQTVQRAVSVNPGK.

Fusion Protein Amino Acid Sequence for IgE/her 2/Neu Immune Complex with Truncated her 2/Neu Domains III and IV (SEQ ID NO. 2):

MANKHLSLSLFLVLLGLSASLASGARVCYGLGMEHLREVRAVTSANI
QEFAGCKKIFGSLAFLPESFDGDPASNTAPLQPEQLQVFETLEEITG
YLYISAWPDSLPDLSVFQNLQVIRGRILHNGAYSLTLQGLGISWLGL
RSLRELGSGLALIHHNTHLCFVHTVPWDQLFRNPHQALLHTANRPED
ECVGEGLACHQLCARGHCWGPGPTQCVNCSQFLRGQECVEECRVLQG
LPREYVNARHCLPCHPECQPQNGSVTCFGPEADQCVACAHYKDPPFC
VARCPSGVKPDLSYMPIWKFPDEEGACQPCPINCTHSCVDLDDKGCP
AGGSGGSGGSGGSEVQLVESGGGLVQPGGSLRLSCAASGFNIKDTYI
HWVRQAPGKGLEWVARIYPTNGYTRYADSVKGRFTISADTSKNTAYL
QMNSLRAEDTAVYYCSRWGGDGFYAMDYWGQGTLVTVSSASTQSPSV
FPLTRCCKNIPSNATSVTLGCLATGYFPEPVMVTWDTGSLNGTTMTL
PATTLTLSGHYATISLLTVSGAWAKQMFTCRVAHTPSSTDWVDNKTF
SVCSRDFTPPTVKILQSSCDGGGHFPPTIQLLCLVSGYTPGTINITW
LEDGQVMDVDLSTASTTQEGELASTQSELTLSQKHWLSDRTYTCQVT
YQGHTFEDSTKKCADSNPRGVSAYLSRPSPFDLFIRKSPTITCLVVD
LAPSKGTVNLTWSRASGKPVNHSTRKEEKQRNGTLTVTSTLPVGTRD
WIEGETYQCRVTHPHLPRALMRSTTKTSGPRAAPEVYAFATPEWPGS
RDKRTLACLIQNFMPEDISVQWLHNEVQLPDARHSTTQPRKTKGSGF
FVFSRLEVTRAEWEQKDEFICRAVHEAASPSQTVQRAVSVNPGK.

Fusion Protein Amino Acid Sequence for Barley Alpha Amylase Signal Peptide (BASP) (SEQ ID NO. 3)

MANKHLSLSLFLVLLGLSASLASG.

Fusion Protein Amino Acid Sequence for her 2/Neu with Full Ectodomain (SEQ ID NO. 4)

TQVCTGTDMKLRLPASPETHLDMLRHLYQGCQVVQGNLELTYLPTNA
SLSFLQDIQEVQGYVLIAHNQVRQVPLQRLRIVRGTQLFEDNYALAV
LDNGDPLNNTTPVTGASPGGLRELQLRSLTEILKGGVLIQRNPQLCY
QDTILWKDIFHKNNQLALTLIDTNRSRACHPCSPMCKGSRCWGESSE
DCQSLTRTVCAGGCARCKGPLPTDCCHEQCAAGCTGPKHSDCLACLH
FNHSGICELHCPALVTYNTDTFESMPNPEGRYTFGASCVTACPYNYL
STDVGSCTLVCPLHNQEVTAEDGTQRCEKCSKPCARVCYGLGMEHLR
EVRAVTSANIQEFAGCKKIEGSLAELPESFDGDPASNTAPLQPEQLQ
VFETLEEITGYLYISAWPDSLPDLSVFQNLQVIRGRILHNGAYSLTL
QGLGISWLGLRSLRELGSGLALIHHNTHLCFVHTVPWDQLFRNPHQA
LLHTANRPEDECVGEGLACHQLCARGHCWGPGPTQCVNCSQFLRGQE
CVEECRVLQGLPREYVNARHCLPCHPECQPQNGSVTCFGPEADQCVA
CAHYKDPPFCVARCPSGVKPDLSYMPIWKFPDEEGACQPCPINCTHS
CVDLDDKGCPA.

Fusion Protein Amino Acid Sequence for her 2/Neu with Truncated Ectodomain Comprising Domains III and IV (SEQ ID NO. 5)

ARVCYGLGMEHLREVRAVTSANIQEFAGCKKIFGSLAFLPESFDGDP
ASNTAPLQPEQLQVFETLEEITGYLYISAWPDSLPDLSVFQNLQVIR
GRILHNGAYSLTLQGLGISWLGLRSLRELGSGLALIHHNTHLCFVHT
VPWDQLFRNPHQALLHTANRPEDECVGEGLACHQLCARGHCWGPGPT
QCVNCSQFLRGQECVEECRVLQGLPREYVNARHCLPCHPECQPQNGS
VTCFGPEADQCVACAHYKDPPFCVARCPSGVKPDLSYMPIWKFPDEE
GACQPCPINCTHSCVDLDDKGCPA.

Fusion Protein Amino Acid Sequence for Linker Peptide Tether (SEQ ID NO. 6)

GGSGGSGGSGGS.

Fusion Protein Amino Acid Sequence for Trastuzumab-IgE c-Heavy Chain (PDB:60GE_E) (SEQ ID NO. 7)

EVQLVESGGGLVQPGGSLRLSCAASGFNIKDTYIHWVRQAPGKGLEW
VARIYPTNGYTRYADSVKGRFTISADTSKNTAYLQMNSLRAEDTAVY
YCSRWGGDGFYAMDYWGQGTLVTVSSASTQSPSVFPLTRCCKNIPSN
ATSVTLGCLATGYFPEPVMVTWDTGSLNGTTMTLPATTLTLSGHYAT
ISLLTVSGAWAKQMFTCRVAHTPSSTDWVDNKTFSVCSRDFTPPTVK
ILQSSCDGGGHFPPTIQLLCLVSGYTPGTINITWLEDGQVMDVDLST
ASTTQEGELASTQSELTLSQKHWLSDRTYTCQVTYQGHTFEDSTKKC
ADSNPRGVSAYLSRPSPFDLFIRKSPTITCLVVDLAPSKGTVNLTWS
RASGKPVNHSTRKEEKQRNGTLTVTSTLPVGTRDWIEGETYQCRVTH
PHLPRALMRSTTKTSGPRAAPEVYAFATPEWPGSRDKRTLACLIQNF
MPEDISVQWLHNEVQLPDARHSTTQPRKTKGSGFFVFSRLEVTRAEW
EQKDEFICRAVHEAASPSQTVQRAVSVNPGK.

Fusion Protein Amino Acid Sequence Trastuzamab-IgE Light Chain (PDB:60GE_E). KEGG Entry D03257 214 Amino Acids 23.47 KDa FASTA (SEQ ID NO. 8)

DIQMTQSPSSLSASVGDRVTITCRASQDVNTAVAWYQQKPGKAPKLL
IYSASFLYSGVPSRFSGSRSGTDFTLTISSLQPEDFATYYCQQHYTT
PPTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC.

Constructing plasmid vectors for expression of target protein candidates: In another embodiment, the disclosure enables the design of DNA coding sequences for the proteins with codons are optimized for expression in a plant subject Nicotiana benthamiana, with restriction sites to facilitate gene cloning, given a set of candidates Her-2-IgE fusion proteins (See FIG. 15 and FIG. 16). One having ordinary skill in the art will recognize that the genes can be chemically synthesized by a commercial platform that specializes in the synthesis of single- and double-stranded DNA fragments and cloned genes for synthetic biology, such as Ultramer DNA Oligos. In another embodiment, the genes are inserted into geminiviral expression vectors that include cassettes for both heavy chain and light chain of IgE, with the Her-2 gene fused in frame via a linker to either the heavy chain or light chain. In another embodiment, the expression construct sequences are verified and used to transform Agrobacterium tumefaciens EHA105 and select and bank clones verified by PCR and restriction digest of plasmids.

Expression and testing of target protein candidates for selection of the optimal construct: In another embodiment, agro-infiltration of plasmid vectors into leaves of N. benthamiana enable recombinant expression of the target proteins. The plants must be monitored daily, and leaves collected every 3, 4, and 5 days after infiltration to optimize timing. In another embodiment, soluble proteins are extracted from leaves, and resolved by SDS-PAGE under either reducing or non-reducing conditions. Reducing conditions show the heavy chain and light chain polypeptides at the expected molecular weights (depending on type of Her-2 fusion), while non-reducing conditions maintain disulfide-bonds of correctly assembled high-molecular weight complexes (H₂L₂). In another embodiment, gels are stained for total protein, and separate gels are electro-transferred to PVDF membranes for western blotting. The blots are probed with antibodies specific for IgE heavy chain, light chain, and Her-2 to verify that all components are present in the high-MW complexes. Examination of gels and blots provide evidence of proteolytic degradation, especially since the composition or length of the linker peptides affects the stability of the fusion protein in vivo as well as during protein extraction and analysis. One of ordinary skill would recognize that an optimal construct can be selected based on analysis of these data.

Demonstration of correct binding of the IgE variable regions with the Her-2 antigenic site can be achieved through examination of molecular structures by methods such as X-ray crystallography, cryo-electron microscopy, or nuclear magnetic resonance studies. [Hao, (2019)]

Purifying optimal target protein products: Purification of a selected target protein can be achieved using protein A-affinity chromatography after filtration of soluble leaf extracts to remove bacteria-size particles. Following low-pH elution and immediate neutralization, size-exclusion chromatography is used to purify the full-size, correctly assembled complexes. If concentration of the target protein is needed, centrifugal concentrators can be used to obtain the appropriate protein content. One of ordinary skill would recognize that the final product from the centrifuge can be assayed by SDS-PAGE and western blot to confirm molecular integrity.

### Results

Using modified IgE to produce an IgE antibody/Her-2 Immune Complex: Targeted oncogen consists of the truncated ectodomain of human Her-2. The truncated ectodomain enhances immune complex stability. In some embodiments, domains III and IV are separated from domains I and II of the full length ectodomain of Human Her-2. In some embodiments, domains III and IV are attached to the complimentary regions of the N-terminus variable regions of the IgE molecule with a fusion sequence of at least 10 amino acids.

The immune complex is designed to ensure stability during processing as an antigen/antibody immune complex capable of negotiating a dysregulated immune environment intact and engaging the targeted CD23 low affinity receptor on B-cells as well as the FcεRI high affinity IgE receptor.

As shown in FIG. 14, the IgE/Her 2/neu immunocomplex binds human CD23 (low affinity receptor) with greater affinity than human IgE alone. Targeting glycosylation sites GS1-GS7 on the IgE antibody with >90% GlcNAc terminating glycoforms results in an immune complex with higher affinity for the CD23 low affinity IgE receptor than the FcεRI high affinity IgE receptor. Engaging the CD23 receptor is essential for unique immune complex processing by the B cell. [Montero-Morales et al. (2017), Jegouzo et al. (2019), Acharya et al. (2010)].

Several implementations have been described. Nevertheless, it will be understood that various modifications may be made without departing from the spirit and scope of the disclosure. For example, various forms of the methods described above may be used, with steps re-ordered, added, or removed. Accordingly, other implementations are within the scope of the following claims.

What is claimed is:

1. A stabilized artificial immunocomplex comprising a protein molecule genetically fused to an isolated antibody.

2. The immunocomplex of claim 1, wherein the protein molecule comprises a tumor-specific oncogenic Her-2/neu molecule derived from a tumor sample extracted from a subject.

3. The immunocomplex of claim 2, wherein the tumor-specific oncogenic Her-2/neu molecule comprises a Her-2/neu molecule with a full ectodomain.

4. The immunocomplex of claim 3, wherein the Her-2/neu molecule is genetically fused to the isolated antibody with a tether comprising at least ten (10) amino acid residues.

5. The immunocomplex of claim 3, wherein the Her-2/neu molecule is genetically fused to the isolated antibody ex vivo.

6. The immunocomplex of claim 2, wherein the tumor-specific oncogen Her-2/neu molecule comprises a Her-2/neu molecule with a truncated ectodomain comprising domains III and IV.

7. The immunocomplex of claim 6, wherein the Her-2/neu molecule is genetically fused to the isolated antibody at its domain III or its domain IV with a tether comprising at least ten (10) amino acid residues.

8. The immunocomplex of claim 6, wherein the Her-2/neu molecule is genetically fused to the isolated antibody ex vivo.

9. The immunocomplex of claim 2, wherein the tumor-specific oncogen Her-2/neu molecule is genetically fused to an N-terminus of the isolated antibody at a C-terminus of its domain III or domain IV.

10. The immunocomplex of claim 1, wherein the isolated antibody comprises an IgE class antibody.

11. The immunocomplex of claim 10, wherein the isolated antibody comprises an epsilon heavy chain variable region and a light chain variable region that are similar to those regions found in the monoclonal antibody trastuzumab.

12. The immunocomplex of claim 10, wherein the isolated antibody comprises an Fc region that binds to a FcεRI (CD23) receptor on a B cell, or a FcεRI receptor on a dendritic cell, or a FcεRII receptor on a dendritic cell.

13. The immunocomplex of claim 10, wherein the isolated antibody is a recombinant monoclonal antibody.

14. The immunocomplex of claim 10, wherein the isolated antibody is a humanized or chimeric antibody.

15. The immunocomplex of claim 10, wherein the isolated antibody is an antibody fragment that binds Her-2/neu.

16. The immunocomplex of claim 10,
wherein the immunocomplex is mono-epitopic;
wherein the immunocomplex has similar binding specificity in each arm of the N-terminus of the isolated antibody of the immunocomplex;
wherein the immunocomplex has similar tumor-specific oncogen linked to the complimentary determining region (CDR) of each arm of the N-terminus of the isolated antibody of the immune complex.

17. A pharmaceutical composition comprising the immunocomplex of claim 1, and a pharmaceutically acceptable carrier.

18. A method for treating a malignant breast cancer tumor in a subject, comprising administering to the subject a therapeutically effective amount of the pharmaceutical composition of claim 17, wherein the malignant breast cancer tumor expresses Her-2/neu.


SEQUENCE LISTING (8 sequences)

SEQ ID NO: 1 - protein, 1206 aa, Nicotiana benthamiana
MANKHLSLSLFLVLLGLSASLASGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAGGSGGSGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 2 - protein, 890 aa, Nicotiana benthamiana
MANKHLSLSLFLVLLGLSASLASGARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAGGSGGSGGSGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTQSPSVFPLTRCCKNIPSNATSVTLGCLATGYFPEPVMVTWDTGSLNGTTMTLPATTLTLSGHYATISLLTVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 3 - protein, 24 aa, Nicotiana benthamiana
MANKHLSLSLFLVLLGLSASLASG

SEQ ID NO: 4 - protein, 622 aa, Nicotiana benthamiana
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPA

SEQ ID NO: 5 - protein, 306 aa, Nicotiana benthamiana
ARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPA

SEQ ID NO: 6 - protein, 12 aa, Nicotiana benthamiana
GGSGGSGGSGGS

SEQ ID NO: 7 - protein, 890 aa, Nicotiana benthamiana; identical to SEQ ID NO: 2

SEQ ID NO: 8 - protein, 214 aa, Nicotiana benthamiana
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC